(19) United States
(12) Patent Application Publication
(10) Pub. No.: US 2026/0301911 A1
(43) Pub. Date: Oct. 1, 2026

(54) MODULAR TREATMENT SYSTEM

(71) Applicant: MAS Innovation (Private) Limited, Battaramulla (LK)

(72) Inventors: Shahen MANUEL, Battaramulla (LK); Lakitha JAYASEKERA, Battaramulla (LK); Sulakshi RAJARATNE, Battaramulla (LK); Savinda GUNAWARDENA, Battaramulla (LK); Waruna WIJESEKARA, Battaramulla (LK); Dilum MALINGA, Battaramulla (LK); Mihan ZAIN, Battaramulla (LK); Kathesh HANDY, Battaramulla (LK); Isuru GALGAMUWA, Battaramulla (LK)

(73) Assignee: MAS Innovation (Private) Limited, Battaramulla (LK)

(21) Appl. No.: 19/478,714

(22) PCT Filed: Apr. 26, 2024
(86) PCT No.: PCT/SG2024/050272

§ 371 Date: Oct. 24, 2025

(30) Foreign Application Priority Data
Apr. 27, 2023 (SG) 10202301170Y

(51) Int. Cl. G16H 20/30; A61B 5/00

(52) U.S. Cl. CPC G16H 20/30; A61B 5/4836; A61B 5/6802; A61B 5/7475; A61B 2560/0443

(57) ABSTRACT

A modular treatment system comprises a therapeutic module configured to provide a physical therapy to a subject; and a holder module configured to maintain the therapeutic module in a position proximate to the subject, wherein the therapeutic module is detachably couplable to the holder module.

### TECHNICAL FIELD

The present disclosure relates to systems such as pain management systems for applying a treatment to a patient.

### BACKGROUND

A wide variety of physical therapies exist. Currently these physical therapies are applied to subjects by dedicated devices which are limited to the application of a single physical therapy.

### SUMMARY OF THE INVENTION

According to a first aspect of the present disclosure a modular treatment system comprises a therapeutic module configured to provide a physical therapy to a subject; and a holder module configured to maintain the therapeutic module in a position proximate to the subject, wherein the therapeutic module is detachably couplable to the holder module.

In an embodiment, the modular treatment system comprises a plurality of interchangeable therapeutic modules each being detachably couplable to the holder module.

In an embodiment the holder module is a wearable module configured to be worn by the subject. For example, the holder module may be a softgood module such as a garment. In an embodiment, the holder module is ergonomically configured for a part of the body of the subject. As an alternative to being a wearable module, the holder module may be a part of an item such as a chair.

In an embodiment, the holder module comprises a plurality of panels, each being configured to receive a therapeutic module. This allows multiple therapeutic modules to be attached to the holder module

In an embodiment, the therapeutic module comprises a sensor configured to monitor a physical attribute of the subject. The sensor may be configured to monitor a physical attribute of the subject corresponding to the physical therapy provided by the therapeutic module.

In an embodiment, the therapeutic module comprises a power supply configured to provide power for the physical therapy. The power supply may be a battery

In an embodiment, the modular treatment system further comprises a controller configured to control the therapeutic module. The controller may be configured to provide user interface which allows a user to control the therapeutic module. The user interface may allow the user to control multiple therapeutic modules. The controller may be configured to communicate with a database and thereby analyze treatment provided to the subject.

In an embodiment, the therapeutic module comprises a wireless transceiver.

The therapeutic module may configured to provide a combination of the following treatments: Thermotherapy, Photobiomodulation, Pulsed electromagnetic therapy (PEMF), cryotherapy, active compound release, transcutaneous electrical nerve stimulation (TENS), Neuromuscular Electrical Stimulation (NMES), microcurrent electrical nerve stimulation (MENS) lonotophoresis, ultrasound, traction, phonophoresis, thermophoresis, tecar, and compression.

### BRIEF DESCRIPTION OF THE DRAWINGS

In the following, embodiments of the present invention will be described as non-limiting examples with reference to the accompanying drawings in which:

FIG. 1 shows a modular treatment system according to an embodiment of the present invention;

FIG. 2 shows the components of a therapeutic module according to an embodiment of the present invention;

FIG. 3A and FIG. 3B are front and back views of a subject wearing a modular treatment system according to an embodiment of the present invention;

FIG. 4 shows a modular treatment system according to an embodiment of the present invention; and

FIG. 5 shows a modular treatment system according to an embodiment of the present invention.

### DETAILED DESCRIPTION

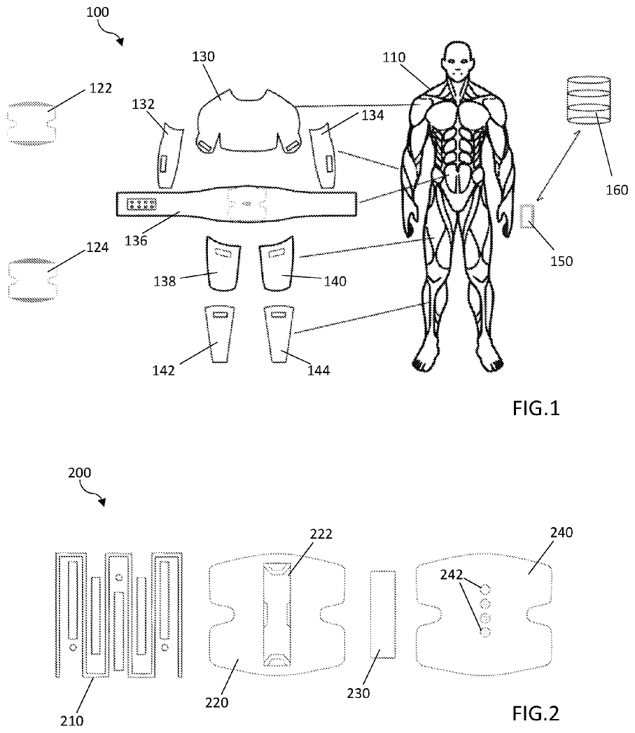

FIG. 1 shows a modular treatment system according to an embodiment of the present invention. As shown in FIG. 1, the pain management system 100 comprises a plurality of therapeutic modules 122 124. The therapeutic modules 122 124 are detachably attachable to multiple softgood form factors designed for the ergonomics of different body parts of a subject 110. As shown in FIG. 1, the softgood elements factors comprise an upper body portion 130, a left arm portion 132, a right arm portion 134, a waist portion 136, a left thigh portion 138, a right thigh portion 140, a left lower leg portion 142 and a right lower leg portion 144.

The therapeutic modules 122 124 are wirelessly coupled to a control panel 150 which is connected to a database 160. The singular control panel provides easy access for the subject 110 where the multiple therapeutic modules capture data and relay it to a software/application regarding treatment, device and quality of life to provide insights based on multiple factors.

While FIG. 1 shows a particular division of the soft good elements, it will be appreciated that different configurations of softgood elements may be used, for example, the softgood elements may be divided into an upper body portion and a lower body portion, or there may be separate leg portions covering both the upper leg and the lower leg of the subject. A user can attach and detach multiple therapeutic modules with different physiological effects to manage pain and conditions where a user will be able to utilize various modules. The multiple softgood factors make it easier for someone who has limited mobility and flexibility to wear the product, especially on hard to reach parts of the body. A user can control multiple active therapeutic modules at the same time through the form of a software/application which will gather data from each of the modules and their unique biometrics providing insight into how the therapy is affecting each part of the body.

Each therapeutic module is implemented as a combination of technologies that can be used to provide short and long term pain relief, where the complimentary technologies have a multiplier effect on a user and underlying system subjectively and quantitatively. Each of these modules can be attached and detached onto any one of the softgood units that cater to multiple body parts. While each combination is unique they can all be utilized by the embedded the control panel on any of the softgood forms. This gives users the flexibility of dealing with their condition, while still making the products easy to access.

Each therapeutic module uses a sensor, unique to each of the potential combination therapies in each module, to capture biometric data, treatment information, device (module) and quality of life. Each module will provide multiple different data sets unique to the treatment, location and starting conditions. Through data gathered across multiple body parts and locations, the system is able to provide feedback through an interface, where a user, clinician, researcher etc. can use the information to gain insights into improvement, efficiency, changes, compliance etc.

FIG. 2 shows the components of a therapeutic module according to an embodiment of the present invention. In the embodiment shown in FIG. 2, the therapeutic module 200 is a Thermotherapy/Photobiomodulation therapy module. As shown in FIG. 2, the therapeutic module 200 comprises a Thermotherapy/Photobiomodulation therapy element 210, a module front 220 with an electronic casing cavity 222, an electronic casing 230 and a module back 240 with snaps 242.

The Thermotherapy/Photobiomodulation therapy element 210 comprises a heating element for applying heat for thermotherapy and a lighting element light of either a specific frequency or a range of frequencies. The electronic casing 230 contains electronic circuitry for controlling the Thermotherapy/Photobiomodulation therapy element 210 and for communicating with the control panel 150. The electronic casing 230 also contains a power supply such as a battery to power the therapeutic module 200. The module back 240 is shaped to fit against the body of the patient. The snaps 242 allow the therapeutic module 200 to be detachably coupled to one of the softgood elements.

The control panel is provided with a software/application which can be used to control the multiple modules across multiple body parts from a single device, each providing a different form of combination therapy to the body; this system will also act as the main interface for users to see the outputs of their treatments, be in the form of graphs, notifications, tables, images etc.

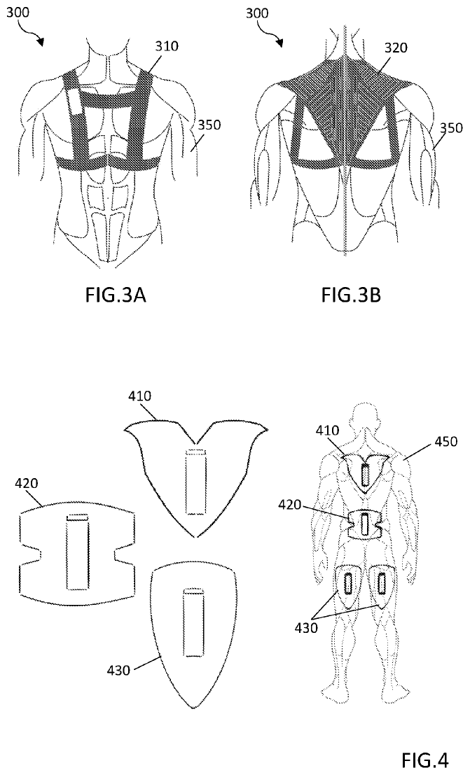

FIG. 3A and FIG. 3B are front and back views of a subject wearing a modular treatment system according to an embodiment of the present invention. As shown in FIG. 3A, the front of the modular treatment system 300 comprises straps 310 which run over the shoulders of the subject 350 and under the arms of the subject 350. As shown in FIG. 3B, the back of the modular treatment system comprises a detachable treatment module 320 which covers the shoulders and upper back of the subject 350.

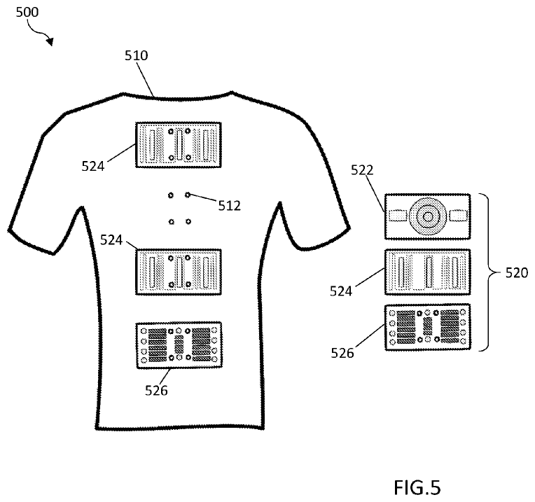

FIG. 4 shows a modular treatment system according to an embodiment of the present invention. In this embodiment the shape of the treatment modules varies with their position on the body of the subject 450. The surface of the treatment modules may also be configured to fit against the body of the subject. A first treatment module 410 is configured to fit the upper back of the subject 450. A second treatment module 420 is configured to fit the lower back of the subject 450. A third treatment module 430 is configured to fit the thighs of the subject 450.

FIG. 5 shows a modular treatment system according to an embodiment of the present invention. As shown in FIG. 5, the modular treatment system 500 comprises a garment 510 and a plurality of treatment modules 520. The garment 510 is in the form of a T-shirt and has a plurality of fasteners 512 arranged running down the back of the subject. The fasteners 512 allow the treatment modules 520 to be attached in positions on the back of the subject. In this example, the treatment modules 520 comprise a PEMF and ultrasound therapy module 522. A heat and light therapy module 524, and a cryotherapy and haptics module 526. As shown in FIG. 5, two heat and light therapy modules 524 and a cryotherapy and haptics module 526 are attached to the garment 510. To change the treatment applied to the subject, the treatment modules may be detached and different treatment modules attached to the fasteners 512.

While the above description relates to apparel softgood modules, embodiments can be used in other systems such as: Where the softgood factor can be added to ergonomic systems such as office chairs and car seats to allow for a therapeutic module to be attached to a certain area and for a user to control according to their therapeutic need, while capturing data from a device that may be currently attached to the car seat, and from one which is providing therapeutic relief to the forearm. In this case as such the user can detach the module from the car seat and attach it to a softgood form for the thigh and utilize it through there. Stating that the same module is now on the thigh and the data is relevant for a specific part of the body.

The system consists of a softgood product, therapeutic module and a software/mobile application. The softgood element, which may be in the form of an article of clothing, apparel etc. can be worn on a number of body parts including but not limited to: Lower back, upper back, upper back and neck, shoulder, knee, ankle, wrist, elbow, forearm, thight, quads, calves, chest etc. This softgood piece consists of an embedded control panel and connectors that are compatible with every therapeutic module released.

The therapeutic module is an element that consists of physical therapy combinations that can be used to treat pain and conditions, where the combinations are selected based on how they interact with one another to give more benefits than each modality in of by itself.

This module will have connectors that which can snap/connect to any of the softgood element body options, allowing for a user to plug/snap to any therapeutic module to the softgood element and control it via the button panel located at the front. Each therapeutic element will consist of a sensor that is selected to give outputs based on of the effect of the therapy on the body. The therapeutic module will consist of a rechargeable battery/power supply, control, power or control PCB, sensors and processing system, an element of support (example. Back posture support through the use of a semi-rigid or malleable material) and a minimal UX/UI. Therapeutic combinations may consist of combinations (two or more) such as, but not limited to:
Thermotherapy and Photobiomodulation
Pulsed electromagnetic therapy (PEMF) and cryotherapy
Thermotherapy and active release compounds
Transcutaneous electrical nerve stimulation (TENS) and Photobiomodulation
Cryotherapy and microcurrent electrical nerve stimulation (MENS)

Other modalities include but are not limited to: Ultrasound, TENS, microcurrent electrical nerve stimulation (MENS), Neuromuscular electrical stimulation (NMES), PEMF, lontophoresis, Thermophoresis, Magnetotherapy etc.

The software/mobile application element, will consist of a base user interface that can be used by a user or additional stakeholders. The application will allow for a user to not only control multiple therapy modules at once, but also allow the user to access more advanced functions of the modules. The application will pass information from sensors and surveys into a database where aspects of data science and machine learning can be used to understand both insights into a user pain across single and multiple regions on a body, the effects of multiple therapies across each of these areas, the effect on a user's quality of life and give predictive information on how best to use the systems and multiple modules. This information can be sent to multiple stakeholders to provide key insights on a particular user in the case of a doctor patient plan. This information can provide high level insights for research groups and clinicians.

The softgood element provides a means for a user to easily attach therapeutic systems to a particular body part and utilize each separate system through a simple user interface. This system can be incorporated into other elements such as chairs, plane seats, cars seats etc. This can be added as an attachment to current therapeutic systems such as existing compression systems, heating blankets, hospital beds etc.

The therapeutic element brings the aspect of modularity allowing a user to quickly switch out the physiological effect on a system based on the desired outcome.

Example: A user who has an acute injury will use the cryotherapy and PEMF combo, as they progress into a subacute phase they may switch the module in the same region to one which is thermotherapy and photobiomodulation.

The software/application will take information from the various sensors and provide a user with an interface, data analysis and report generation to reflect the therapies used on a person along with how utilizing and not utilizing the product may affect the user. The application will also act as a gateway for a user to control multiple active therapeutic modules on a person. This gateway will capture information from each of the modules and tie it to the location the module is used on and how effective it is. In the example of a person switching between therapies the mobile system can be used to access more advanced treatment functions (more detailed or personalized profiles), allow for a user to see how their recovery is going, which treatments work best at which times, the improvement to quality of life etc.

While the above description is focused on managing conditions, embodiments of the invention could be used as a means of data gathering for researchers, a way for doctors to check on compliance of a user, a way of physiotherapists to improve recovery, it can be used as a rehabilitation method and as ways to improve wellness through external stimulation. It can be used to gather insights on conditions and understand trends, consumer pain points, effects of quality of life across large demographics, market growth etc. The pain management system allows for a user to seamlessly utilize multiple systems across a single platform. It makes it easy for a user to wear, use and understand the effects of these therapies on them.

Whilst the foregoing description has described exemplary embodiments, it will be understood by those skilled in the art that many variations of the embodiments can be made within the scope and spirit of the present invention.

1. A modular treatment system comprising:
a therapeutic module configured to provide a physical therapy to a subject; and
a holder module configured to maintain the therapeutic module in a position proximate to the subject,
wherein the therapeutic module is detachably couplable to the holder module.

2. The modular treatment system according to claim 1, comprising a plurality of interchangeable therapeutic modules each being detachably couplable to the holder module.

3. The modular treatment system according to claim 1, wherein the holder module is a wearable module configured to be worn by the subject.

4. The modular treatment system according to claim 3, wherein the holder module is ergonomically configured for a part of the body of the subject.

5. The modular treatment system according to claim 1, wherein the holder module comprises a plurality of panels, each being configured to receive a therapeutic module.

6. The modular treatment system according to claim 1, wherein the therapeutic module comprises a sensor configured to monitor a physical attribute of the subject.

7. The modular treatment system according to claim 6, wherein the sensor is configured to monitor a physical attribute of the subject corresponding to the physical therapy provided by the therapeutic module.

8. The modular treatment system according to claim 1, wherein the therapeutic module comprises a power supply configured to provide power for the physical therapy.

9. The modular treatment system of claim 8, wherein the power supply is a battery.

10. The modular treatment system according to claim 1, further comprising a controller configured to control the therapeutic module.

11. The modular treatment system according to claim 10, wherein the controller is configured to provide user interface which allows a user to control the therapeutic module.

12. The modular treatment system according to claim 11, wherein the user interface allows the user to control multiple therapeutic modules.

13. The modular treatment system according to claim 11, wherein the controller is configured to communicate with a database and thereby analyze treatment provided to the subject.

14. The modular treatment system according to claim 1, wherein the therapeutic module comprises a wireless transceiver.

15. The modular treatment system according to claim 1, wherein the therapeutic module is configured to provide a combination of the following treatments: Thermotherapy, Photobiomodulation, Pulsed electromagnetic therapy (PEMF), cryotherapy, active compound release, transcutaneous electrical nerve stimulation (TENS), Neuromuscular Electrical Stimulation (NMES), microcurrent electrical nerve stimulation (MENS) Ionotophoresis, ultrasound, traction, phonophoresis, thermophoresis, tecar, and compression.
